(19) United States
(12) Patent Application Publication
(10) Pub. No.: US 2025/0281481 A1
(43) Pub. Date: Sep. 11, 2025

(54) NALTREXONE FOR BOOSTING THE THERAPEUTIC UTILTY OF 5-HT RECEPTOR AGONISTS

(71) Applicant: LDN Pharma Limited, London (GB)

(72) Inventors: Wai Liu, London (GB); Ian Thompson, London (GB); Francis Hood, London (GB)

(21) Appl. No.: 18/572,637

(22) PCT Filed: Jun. 23, 2022
(86) PCT No.: PCT/GB2022/051606

§ 371 Date: Dec. 20, 2023

(30) Foreign Application Priority Data
Jun. 23, 2021 (GB) 2109018.8

(51) Int. Cl. A61K 31/485; A61K 9/20; A61K 31/593; A61K 45/06

(52) U.S. Cl. CPC A61K 31/485; A61K 9/2009; A61K 9/2013; A61K 9/2027; A61K 9/2054; A61K 9/2059; A61K 31/593; A61K 45/06

(57) ABSTRACT

A single unit oral dose pharmaceutical composition for use as a medicament for separate, sequential or simultaneous administration with an agonist of a 5-hydroxytryptamine (5-HT) receptor;
wherein the single unit oral dose pharmaceutical composition comprises a first active ingredient and a second active ingredient;
wherein the first active ingredient is for absorption in the gastrointestinal tract from the oesophagus onwards and the second active ingredient is for absorption in the oral cavity; and
wherein the first active ingredient is naltrexone in an amount of 0.01 to 10 mg and the second active ingredient is calcitriol in an amount of 80 to 200 ug.

### CROSS REFERENCE TO RELATED APPLICATIONS

This application is a U.S. National Phase application filed under 35 U.S.C. § 371 and claims priority to International Application No. PCT/GB2022/051606, filed on Jun. 23, 2022, which application claims priority to Great Britain Application No. 2109018.8, filed Jun. 23, 2021, the disclosures of which are incorporated herein by reference.

### FIELD OF THE INVENTION

The present invention is directed to a single unit oral dose pharmaceutical composition and an agonist of a 5-hydroxytryptamine (5-HT) receptor, for use in the treatment of various disorders, such as cognitive or neurological disorders, or autoimmune diseases.

### BACKGROUND OF THE INVENTION

Serotonin (5-hydroxytryptamine; 5-HT) is a biogenic amine with a complex and multifaceted biological function. The main receptors and their subtypes are 5-HT1 (5-HT1A, 5-HT1B, 5-HT1D, 5-HT1E and 5-HT1F), 5-HT2 (5-HT2A, 5-HT2B and 5-HT2C), 5-HT3, 5-HT4, 5-HT5 (5-HT5A, 5-HT5B), 5-HT6 and 5-HT7. The receptors are all G-protein coupled receptors (GPCR) except 5-HT3 receptors, which are ionic channels.

Drugs which are capable of either selectively stimulating or inhibiting these receptor subtypes have therapeutic potential in a wide variety of disease conditions. Specifically, agonists of 5-HT receptors are known to be useful in the treatment of various neurological and cognitive disorders, and autoimmune diseases.

Meltzer et. al, Curr Pharm Biotechnol. (2012), 13(8): “Serotonin receptors as targets for drugs useful to treat psychosis and cognitive impairment in schizophrenia” discloses the use of 5-HT2C agonists in the treatment of psychosis and cognitive impairment.

Dukat, Current Medicinal Chemistry, Vol. 4, Issue 2, 2004 “5-HT3 serotonin receptor agonists: a pharmacophoric journey” discloses that 5-HT3 receptor agonists are used in the treatment of migraine and numerous central nervous disorders.

Pithadia et. al.; J Clin Med Res 2009; 1(2); 72-80, “5-Hydroxytryptamine Receptor Subtypes and their Modulators with Therapeutic Potentials” discloses 5-HT1D receptor agonists for use in the treatment of migraine; 5-HT1A receptor agonists for use in the treatment of anxiety; and 5-HT4 agonists for use in the treatment of irritable bowel syndrome.

However, there is an ongoing need to develop supportive treatments that enhance the safety and/or efficacy of the treatment of conditions with 5-HT receptor agonists. For example, it would be desirable to improve the therapeutic efficacy of such agonists of 5-HT receptors, by treating the patient in combination with another pharmaceutical composition.

Furthermore, there is a need to administer drugs to patients in a manner that maximises therapeutic effect and minimises any adverse effects or issues. Oral dosage forms such as tablets or capsules to be swallowed by the patient are commonly used. However, one major limitation is lack of bioavailability of the active ingredient through this means of administration (i.e. by swallowing).

Thus, there is a need to develop treatments that boost the therapeutic utility of 5-HT receptor agonists, and maximise the bioavailability of the active ingredients.

### SUMMARY OF THE INVENTION

According to a first aspect of the invention, there is provided a single unit oral dose pharmaceutical composition for use as a medicament for separate, sequential or simultaneous administration with an agonist of a 5-hydroxytryptamine (5-HT) receptor;
wherein the single unit oral dose pharmaceutical composition comprises a first active ingredient and a second active ingredient;
wherein the first active ingredient is for absorption in the gastrointestinal tract from the oesophagus onwards and the second active ingredient is for absorption in the oral cavity; and
wherein the first active ingredient is naltrexone in an amount of 0.01 to 10 mg and the second active ingredient is calcitriol in an amount of 80 to 200 ug.

### DETAILED DESCRIPTION OF THE INVENTION

The inventors discovered that a single unit oral dose pharmaceutical composition wherein the single unit oral dose pharmaceutical composition comprises a first active ingredient and a second active ingredient;
wherein the first active ingredient is for absorption in the gastrointestinal tract from the oesophagus onwards and the second active ingredient is for absorption in the oral cavity; and
wherein the first active ingredient is naltrexone in an amount of 0.01 to 10 mg and the second active ingredient is calcitriol in an amount of 80 to 200 ug
is optimised for delivery of its actives for therapeutic utility, when used as an agent that boosts the therapeutic utility of a 5-HT receptor agonist.

The inventors of the present invention have discovered that naltrexone, an orally-administered opioid antagonist with the chemical name morphinan-6-one,17-(cyclopropylmethyl)-4,5-epoxy-3,14-dihydroxy-(5α), increases serotonin (5-HT) receptor levels and can therefore be used to aid the effectiveness of agonists of 5-HT receptors. This has been demonstrated in the examples of the present application where it can be seen that both higher dose and low dose naltrexone increase the levels of mRNA for one or more of 5-HT receptor proteins.

It has also been found that the combination of naltrexone and calcitriol in a single unit oral dose can be administered to patients as part of a treatment involving 5-HT receptor agonists, irrespective of renal or liver function. The benefit of this is that the patients can be prescribed the single unit dose without the requirement for pre-screening for renal or liver function/dysfunction. The clinician therefore has confidence that an adequate dose of the medicaments is being prescribed with minimal potential side-effects.

The inventors have realised the benefit of combining low doses of naltrexone and calcitriol, in forms that provide different absorption rates, such that the components acting in a synergistic manner to aid the desired therapeutic effect.

The single unit oral dose of the present invention allows for different, optimised routes of delivery of the two active ingredients: CCT is absorbed in the oral cavity and naltrexone is absorbed beyond the oral cavity i.e. in the gastrointestinal tract from the oesophagus onwards after being swallowed by the patient. The inventors have found that this results in improved bioavailability of the active ingredients.

The inventors have achieved this improved bioavailability in a single unit dose, which is simple and convenient for patients to take and therefore leads to improved patient compliance.

Calcitriol is a metabolite of vitamin D. The normal metabolism process of vitamin D is that it is first converted into calcifediol (CCD) in the liver and then may be converted to calcitriol (CCT) in the kidneys. CCD has a long half-life and persists in the blood. However, when CCT is needed, CCD is converted to CCT in the kidneys. This essentially means that the concentration of CCT is kept relatively constant due to tightly-controlled regulation by the body. Typically, the plasma concentration is maintained around 60 pg/ml. Increasing the supplementation of vitamin D does not tend to significantly increase the concentration of CCT due to this regulation process.

Certain patients being treated with 5-HT receptor agonists may have impaired renal and liver function. The patient may have elevated CYP enzymes which means vitamin D metabolism is compromised. Specifically, CCT cannot be converted from its precursor CCD as it is unable to be converted from vitamin D.

Therefore, the present inventors identified that it would be desirable to administer CCT specifically to patients with impaired renal and liver function, rather than administer vitamin D.

However, the present inventors noted that, when swallowed by the patient, for example in a tablet, CCT has variable bioavailability which means plasma concentrations can vary. This variability means that the desired dosage may not be achieved. One way to mitigate this problem is to increase the dose per tablet to give the top-end of the dose. However, by doing this, there would be a danger of causing renal damage from toxicity.

The single unit oral dose of the present invention allows for CCT to be absorbed in the oral cavity which has been found to have a more consistent delivery of CCT. Furthermore, the single unit oral dose of the present invention allows for naltrexone to be absorbed beyond the oral cavity i.e. in the gastrointestinal tract from the oesophagus onwards which results in optimum delivery of naltrexone.

Thus the present inventors have identified a composition that is optimised for delivery of its actives, particularly in combination with the use of 5-HT receptor agonists. The composition can be easily administered to patients as a single unit oral dose, whilst maximising the bioavailability of the active ingredients.

In a first aspect of the invention, there is provided a single unit oral dose pharmaceutical composition for use as a medicament for separate, sequential or simultaneous administration with an agonist of a 5-hydroxytryptamine (5-HT) receptor;
wherein the single unit oral dose pharmaceutical composition comprises a first active ingredient and a second active ingredient;
wherein the first active ingredient is for absorption in the gastrointestinal tract from the oesophagus onwards and the second active ingredient is for absorption in the oral cavity; and
wherein the first active ingredient is naltrexone in an amount of 0.01 to 10 mg and the second active ingredient is calcitriol in an amount of 80 to 200 ug.

As used herein the term “pharmaceutical composition” means, for example, a mixture containing a specified amount of a therapeutic compound or compounds, e.g. a therapeutically effective amount, in a pharmaceutically acceptable carrier to be administered to a mammal, e.g., a human in order to treat a disease.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein the term “single unit oral dose” has its normal meaning in the art. It may also be called an oral dosage form. It may, for example, be a tablet, a coated tablet, a bi-layered tablet, a multi-layered tablet, a caplet, a capsule, a disc, a pellet or a granule.

As used herein, the term “agonist” has its conventional meaning as used in the art. Agonists of the 5-hydroxytryptamine (5-HT) receptor are compounds that activate one or more of the main receptors or their subtypes: 5-HT1 (5-HT1A, 5-HT1B, 5-HT1D, 5-HT1E and 5-HT1F), 5-HT2 (5-HT2A, 5-HT2B and 5-HT2C), 5-HT3, 5-HT4, 5-HT5 (5-HT5A, 5-HT5B), 5-HT6 and 5-HT7. An agonist of a 5-HT receptor is a full or partial agonist of one or more of the following receptors/receptors subtypes: 5-HT1 (5-HT1A, 5-HT1B, 5-HT1D, 5-HT1E and 5-HT1F), 5-HT2 (5-HT2A, 5-HT2B and 5-HT2C), 5-HT3, 5-HT4, 5-HT5 (5-HT5A, 5-HT5B), 5-HT6 and 5-HT7.

As used herein, the terms “concurrent administration” or “concurrently” or “simultaneous”, “sequential” or “separate” mean that administration of the agonist and the single unit oral dose pharmaceutical composition occur as part of the same treatment regimen.

“Simultaneous” administration, as defined herein, includes the administration of the agonist and the single unit oral dose pharmaceutical composition within about 2 hours or about 1 hour or less of each other, even more preferably at the same time.

“Separate” administration, as defined herein, includes the administration of the agonist and the single unit oral dose pharmaceutical composition, more than about 12 hours, or about 8 hours, or about 6 hours or about 4 hours or about 2 hours apart.

“Sequential” administration, as defined herein, includes the administration of the agonist and the single unit oral dose pharmaceutical composition each in multiple aliquots and/or doses and/or on separate occasions. The agonist may be administered to the subject before or after administration of the single unit oral dose pharmaceutical composition.

In a preferred embodiment, the single unit oral dose pharmaceutical composition is to be administered prior to the administration of the agonist, preferably between 1 to 4 days before administration of the agonist.

In a preferred embodiment, the 5-HT receptor is 5-HT receptor subtype 2A, 2B or 2C. A high density of 5-HT2A receptors is found in many cortical areas. These receptors are particularly concentrated in the frontal cortex. 5-HT2A receptors also are found in high density in the claustrum, a region which is connected to the visual cortex; in parts of the limbic system; and in the basal ganglia and the olfactory nuclei. 5-HT2B receptors are found in the central nervous system, such as the subiculum, the substantia nigra and the peripheral nervous system, such as vascular smooth muscle. 5-HT2C receptors are present in high density in the choroid plexus. It has been proposed that 5-HT-induced activation of 5-HT2C receptors could regulate the composition and volume of the cerebrospinal fluid. 5-HT2C receptors also are found throughout the brain, particularly in areas of the limbic system, including the hypothalamus, hippocampus, septum, neocortex and regions associated with motor behaviour, including the substantia nigra and globus pallidus. Agonists of 5-HT2 receptors are known to be useful in the treatment of various neurological and cognitive disorders, such as Alzheimer's disease, dementia, motor neuron disease (MND), depression, psychosis and anxiety.

In a preferred embodiment, the 5-HT receptor is 5-HT receptor subtype 3. 5-HT3 receptors are found in high density in peripheral ganglia and nerves, including the superior cervical ganglion and vagus nerve, as well as in the substantia gelatinosa of the spinal cord. The 5-HT3 receptor is homomeric and belongs to the ligand-gated ion channel superfamily. It is a serotonin-gated cation channel that causes the rapid depolarization of neurons. Agonists of 5-HT3 receptors are known to be useful in the treatment of various neurological and cognitive disorders, such as pain, migraine, treatment-resistant depression, anxiety, depression, psychosis and paranoia.

In a preferred embodiment, the agonist of a 5-HT receptor is selected from the group consisting of psilocybin, psilocin, mescaline, lysergic acid diethylamide (LSD), ketamine, salvinorin A, ibotenic acid, muscimol, N,N-dimethyltryptamine (DMT), 3,4-methylenedioxymethamphetamine (MDMA), methyldiethanolamine, also known as N-methyl diethanolamine (MDEA), 3,4-methylenedioxy amphetamine (MDA), 4-Bromo-2,5-dimethoxyphenethylamine (2C-B); 1-(8-Bromo-2,3,6,7-tetrahydrobenzo-[1,2-b;4,5-b′]difuran-4-yl)2-amino-ethane (2C-B-fly); 4-Ethyl-2,5-dimeth-oxyphenethylamine (2C-E); 4-Ethyl-thio-2,5-dimethoxyphenethylamine (2C-T-2); 4-Ethylthio-2,5-dimethoxy-amphetamine (ALEPH-2); 4-Ethylthio-2,5-dimethoxyphenylbutylamine (4C-T-2); 2,5-Dimethoxy-4-ethoxyamphetamine (MEM); 2,4,5-Trimethox-amphetamine (TMA-2); 3,4,5-Trimethoxamphetamine (TMA); 2,5-Dimethoxy-4-bromo-amphetamine (DOB); 2,5-Dimethoxy-4-iodo-amphetamine (DOI); 2,5-Dimethoxy-4-methylamphetamine (DOM); 2,5-Dimethoxy-4-ethylamphet-amine (DOET); 5-Methoxy-N,N-dimethyl-tryptamine (5-MeO-DMT); N,N-Dipropyltryptamine (DPT); 5-Methoxy-N-methyl-N-isopropyltryptamine (5-MeO-MIPT); N,N-Diisopropyltryptamine (DIPT); 5-Methoxy-N,N-diiso-propyltryptamine (5-MeO-DIPT); 6-Fluoro-N,N-dimethyltryptamine (6-fluoro-DMT); lisuride; ibogaine; cis-2a; RR-2b; SS-2c; 2-Ethyl-5-methoxy-N,N-dimethyltryptamine (EMDT), serotonin hydrochloride, or metabolites thereof and combinations thereof.

In a preferred embodiment, the agonist of a 5-HT receptor is selected from the group consisting of m-chlorophenylbiguanide hydrochloride, N-methylquipazine dimaleate, PSEM 895, quipazine dimaleate, RS56812 hydrochloride, serotonin hydrochloride, SR7227 hydrochloride, 1-phenylbiguanide hydrochloride, cereulide, 2-methyl-5-HT, alpha-methyltryptamine, bufotenin, chlorophenylbiguanide, ibogaine, varenicline, YM-31636, and metabolites thereof. In the present invention, the agonist of a 5-HT receptor may be a serotonin analogue. By “serotonin analogue” it is meant a functional analogue of serotonin i.e. compounds showing chemical and biological similarity to serotonin. This may be achieved by making modifications to serotonin in order to prepare a new molecule with similar chemical and biological properties.

In a preferred embodiment, the agonist of a 5-HT receptor is to be administered at its approved therapeutic dose. An approved therapeutic dose will be apparent to one skilled in the art, and may vary according to age, sex, weight, which the skilled person is capable of determining.

The single unit oral dose pharmaceutical composition comprises naltrexone as the first active ingredient.

As used herein “naltrexone” refers to morphinan-6-one,17-(cyclopropylmethyl)-4,5-epoxy-3,14-dihydroxy-(5α). Its empirical formula is C₂₀H₂₃NO₄ and its molecular weight is 341.41 in the anhydrous form (<1% maximum water content).

The chemical structure of naltrexone is:

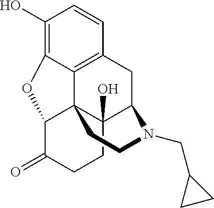

The single unit oral dose pharmaceutical composition comprises naltrexone in an amount of 0.01 to 10 mg. Preferably, the single unit oral dose comprises an amount of 1 mg to 6 mg of naltrexone. In a preferred embodiment, the single unit oral dose pharmaceutical composition comprises naltrexone in an amount of 3 mg to 4.5 mg (i.e. a low dosage). In another preferred embodiment, the single unit oral dose pharmaceutical composition comprises naltrexone in an amount of 5 mg to 10 mg (i.e. a higher dosage amount).

The single unit oral dose pharmaceutical composition is formulated such that the first active ingredient, naltrexone, is for absorption in the gastrointestinal tract from the oesophagus onwards. By this it is meant that naltrexone is absorbed for delivery into the blood stream in the oesophagus, stomach, small intestine, or large intestine, (i.e. the gastrointestinal tract starting from the oesophagus and following its natural course). Naltrexone is not substantially absorbed in the patient's oral cavity.

Absorption of naltrexone into the gastrointestinal tract from the oesophagus onwards is achieved by the patient swallowing the single unit dose.

The single unit oral dose pharmaceutical composition comprises calcitriol as the second active ingredient.

As used herein, “calcitriol” refers to the active metabolite of vitamin D, specifically vitamin D3. Calcitriol is also called 1,25-dihydroxyvitamin-D3 or 1,25-dihydroxycholecalciferol. Its empirical formula is C₂₇H₄₄O₃. Its chemical structure is:

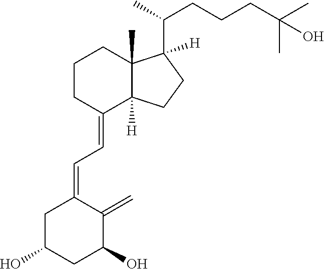 [SMILES: C=C1/C(=C\C=C2/CCC[C@]3(C)[C@@H]([C@H](C)CCCC(C)(C)O)CC[C@@H]23)C[C@@H](O)C[C@@H]1O]

The single unit oral dose pharmaceutical composition comprises calcitriol in an amount of 80 to 200 ug. Preferably, the single unit oral dose comprises 90 to 190 ug, more preferably 100 to 180 ug, most preferably 110 to 170 ug.

The single unit oral dose comprising 80 to 200 ug calcitriol preferably achieves systemic (serum) concentration of calcitriol of 0.1 to 10 ng/ml, more preferably 2 to 8 ng/ml, most preferably 4 to 6 ng/ml.

The single unit oral dose pharmaceutical composition is formulated such that the second active ingredient, calcitriol, is for absorption in the oral cavity. The term oral cavity has its normal meaning in the art and is intended to cover the mouth, the lips, the hard palate, the soft palate, the retromolar trigone, the tongue, gingiva (gums), buccal mucosa (the inner lining of the lips and cheeks), and floor of the mouth under the tongue.

Absorption of calcitriol in the oral cavity may be achieved by the sublingual, sublabial, gingival or buccal route.

In a preferred embodiment, the single unit oral dose pharmaceutical composition according to the present invention comprises the naltrexone in a first formulation and the calcitriol in a second formulation. The first and second formulations are separate from each other, i.e. are not admixed. The first formulation is typically a tablet formulation, and the second formulation is one that permits release and subsequent absorption of the calcitriol in the oral cavity.

The term formulation is intended to include the mixture of the active component(s) with any pharmaceutically acceptable excipients.

In a preferred embodiment, the first formulation comprising naltrexone is formulated as a tablet, a capsule, powder, a disc, a caplet, granules or pellets.

Preferably, the tablet, capsule, powder, disk, caplet, granules or pellets are administered orally and swallowed by the subject. By this it is meant that the subject swallows the tablet, capsule, powder, disk, caplet, granules or pellets which is then metabolised through the traditional route, i.e. in the gastrointestinal tract, from the oesophagus onwards.

The tablet, capsule, powder, disk, caplet, granules or pellets may be provided as a blend of both the naltrexone product and a combination of pharmaceutically acceptable excipients. As used herein, the term “excipient” refers to a pharmaceutically acceptable ingredient that is commonly used in pharmaceutical technology for the preparation of solid oral dosage formulations. Examples of categories of excipients include, but are not limited to, binders, disintegrants, lubricants, glidants, stabilizers, fillers, and diluents. The amount of each excipient used may vary within ranges conventional in the art. The following references which are all hereby incorporated by reference disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2000).

Suitable excipients include magnesium carbonate, magnesium stearate, talc, lactose, lactose monohydrate, sugar, pectin, dextrin, starch, tragacanth, microcrystalline cellulose, methyl cellulose, sodium carboxymethyl cellulose, corn starch, colloidal anhydrous Silica, titanium dioxide, a low-melting wax, cocoa butter, and the like.

The first formulation may comprise components which allow for fast or sustained release of the first active ingredient, naltrexone, in the gastrointestinal tract from the oesophagus onwards. This can be achieved by including pharmaceutically acceptable rate controlling polymers. The rate controlling polymers may be hydrophilic or hydrophobic. Preferably the rate controlling polymers are selected from polyvinyl acetate, cellulose acetate, cellulose acetate butyrate, cellulose acetate propionate, ethyl cellulose, a fatty acid, a fatty acid ester, an alkyl alcohol, microcrystalline cellulose, a poly(meth)acrylate, a poly(ethylene oxide), polyuronic acid salts, cellulose ethers, xanthum gum, tragacanth gum, gum karaya, guar gum, acacia, gellan gum, locust bean gum, alkali metal salts of alginic acid or pectin acid, sodium alginate, potassium alginate, ammonium alginate, hydroxypropyl cellulose, hydroxyl ethyl cellulose, hydroxypropyl ethyl cellulose, carboxyvinyl polymer, polymerised gelatin and/or mixtures thereof.

The second active ingredient, calcitriol, is formulated for absorption in the oral cavity. In a preferred embodiment, the second formulation comprising calcitriol is formulated for absorption by the sublingual route. Absorption in the oral cavity can be achieved according to any known methods. For example, it can be formulated in an orally disintegrating form that disintegrates and dissolves in the mouth without water before swallowing. It may dissolve in this way within 60 seconds or less, preferably less than 10 seconds.

Preferably, the second formulation comprising calcitriol is an orally disintegrating tablet or coating, an orodisperse tablet or coating, a mouth-dissolving tablet or coating, a quick-dissolve tablet or coating, a fast-melt tablet or coating, a rapid-disintegrating tablet or coating; or a freeze-dried wafer.

An orally disintegrating tablet/coating is a solid dosage form that contains medicinal substances and disintegrates rapidly (within seconds) without water when placed on the tongue. The drug is released, dissolved, or dispersed in the saliva.

A quick-dissolving tablet/coating (also known as a fast-dissolving, fast-dissolving multiparticulate, rapid-dissolving, mouth-dissolving, fastmelting, or orodispersing tablets) is a solid dosage form that does not require water for swallowing. The tablet dissolves within 60 seconds when placed in the mouth. The active ingredients are absorbed through mucous membranes in the mouth.

A freeze-dried wafer is a quick-dissolving, thin matrix that contains a medicinal agent (in this case calcitriol) that does not need water for swallowing. The wafer disintegrates instantaneously in the oral cavity and releases calcitriol, which dissolves or disperses in the saliva.

This second formulation may be administered sublingually, sublabially or buccally to the subject by rapidly dissolving in the oral cavity when it comes in contact with saliva. Preferably, it is absorbed sublingually.

The second formulation comprising calcitriol may be formulated according to known methods such as lyophilisation (freeze-drying), cotton candy process, moulding, sublimation and direct compression. In a preferred embodiment, the second formulation comprising calcitriol is formulated as a lyophilised dosage form. Preferably it is a lyophilised liquid solution, suspension or emulsion. The second formulation be achieved according to any known methods for producing dosage forms that dissolve in the oral cavity such as using the following technology: Zydis®, QuickSolv®, Lyoc®, Flashdose®, OraSolve®, Ziplet technology, Frosta®, DuraSolve®, Wowtab®, Durasolv®, Flashtab®, Oraquick®, RapiTab® and Nanomelt® (by Elan).

The present inventors have discovered that the patient absorbing calcitriol in the oral cavity overcomes the problems of variable bioavailability that occurs when calcitriol is swallowed in a tablet. When swallowed, calcitriol has variable bioavailability which means the desired dosage may not be achieved. One way to mitigate this problem is to increase the dose per tablet to give the top-end of the dose. However, by doing this, there would be a danger of causing renal damage from toxicity. The present inventors have discovered that administering calcitriol in the oral cavity allows the desired dosage to be achieved without causing any adverse symptoms associated with renal damage, such as fatigue, loss of appetite or leg swelling. Absorption in the oral cavity also avoids drug metabolism via the stomach and intestines, which means a more efficient delivery of calcitriol.

The second formulation comprising calcitriol may also contain any conventional or pharmaceutically suitable additives suitable for providing absorption in the oral cavity. For example, it may include components that readily dissolve in saliva and contribute to the rapid disintegration in the mouth.

Preferred components may include matrix forming agents and sugars or sugar alcohols. These components are used to give sufficient strength to the unit dose to prevent breakage during removal and packaging, but once placed in the mouth, they allow immediate dissolution of the formulation.

Preferred matrix forming agents include gelatin, starches, modified starches, maltodextrin, cellulose gum, carrageenan gum, hyaluronic acid, pectins, carboxymethyl cellulose sodium, agar, gellan gum, guar gum, tragacanth gum, hydroxypropyl cellulose, hydroxyl propyl methylcellulose, methylcellulose, carbomer, poloxamer, polyacrylic acid, polyvinyl alcohol, alginates and polyglyconic acid or combinations thereof.

Preferred sugar alcohols include mannitol, sorbitol, glycerol, erythritol, maltitol, and lactitol or combinations thereof.

Preferred sugars include trehalose, dextrose, and lactose or combinations thereof.

In a preferred embodiment, the second formulation comprises gelatin and/or mannitol.

Preferably, the second formulation comprising calcitriol is formed by cryogenically freezing a liquid, emulsion or suspension containing calcitriol and optional additives. The frozen units then undergo a lyophilisation process in freeze dryers.

During lyophilisation a multitude of air pockets are created which help absorb the saliva and aid disintegration of the layer, which means the layer can be absorbed quickly in the oral cavity.

In a preferred embodiment, the first formulation comprising naltrexone is in the form of a tablet and the second formulation comprising calcitriol is a lyophilised liquid solution, suspension or emulsion which is attached to the tablet, thus forming a single unit oral dose. The first formulation and second formulation are preferably two separate, distinct parts of the single unit oral dose pharmaceutical composition. The second formulation may be attached to the tablet by any known means. For example, the second formulation may coat the tablet, or it may be formed as a layer, film or wafer in connection with or on the tablet.

The skilled person may use any known method for producing the single unit oral dose comprising a first formulation and a second formulation.

In a preferred embodiment, the single unit oral dose pharmaceutical composition comprises a first formulation comprising naltrexone is in the form of a tablet and a second formulation comprising calcitriol as a lyophilised liquid solution, suspension or emulsion which is attached to the tablet, thus forming a single unit oral dose. In this embodiment, the first formulation comprising naltrexone in the form of a tablet is formulated for delayed release. By delayed release, it is meant that the naltrexone is released after it has been swallowed by the patient. This ensures that naltrexone is predominantly absorbed in the gastrointestinal tract from the oesophagus onwards i.e. in the stomach. Furthermore, the second formulation comprising calcitriol being a lyophilised liquid solution, suspension or emulsion which is attached to the tablet ensures that calcitriol is absorbed in the oral cavity.

Furthermore, in a preferred embodiment, the first formulation comprising naltrexone is formulated for release and/or absorption after the second formulation comprising calcitriol is released and/or absorbed. By this it is meant that the calcitriol is formulated such that it is released and/or absorbed by the patient before the naltrexone is released and/or absorbed by the patient.

According to a further aspect of the invention, there is provided a method of forming a single unit oral dose pharmaceutical composition, comprising the following steps:
Providing a first formulation comprising 0.01 to 10 mg naltrexone;
Providing a second formulation in contact with the first formulation, wherein the second formulation is a solution, suspension or emulsion comprising 80 to 200 ug calcitriol;
Optionally freezing the combination of the first formulation and second formulation;
Lyophilising the combination of the first formulation and the second formulation.

In a preferred embodiment, there is provided a method of forming a single unit oral dose pharmaceutical composition according to the present invention, comprising the steps of:
(i) Providing a first formulation comprising 0.01 to 10 mg naltrexone;
(ii) Compressing the first formulation into a tablet, capsule, powder, disk, caplet, granule or pellet;
(iii) Providing a second formulation comprising 80 to 200 ug calcitriol, wherein the second formulation is a solution, suspension or emulsion;
(iv) Dosing the second formulation into a pre-formed mould and optionally freezing the second formulation:
(v) Adding the first formulation to the preformed mould, such that the first formulation is in contact with the second formulation;
(vi) Lyophilising the combination of the first formulation and the second formulation.

In a preferred embodiment, the first step is formulation of a mixture comprising 0.01 to 10 mg naltrexone. This mixture may be made by any known method, such as dry granulation or wet granulation. This mixture may optionally comprise a filler, a glidant, a disintegrant and/or a lubricant. The mixture is preferably compressed into a tablet capsule, powder, disk, caplet, granule or pellet.

In a next step, the second formulation comprising 80 to 200 ug calcitriol is formulated as a solution, suspension or emulsion. The second formulation optionally comprises a matrix forming agent and/or a sugar or sugar alcohol, preferably mannitol and gelatin.

Preferably, the second formulation is then dosed into a preformed mould. In a next step, the first formulation is attached to the second formulation and the combination of formulations is frozen. This may be achieved by placing the pre-formed tablet in the mould, in contact with the second formulation. The freezing step is preferably carried out rapidly, for example for a period of 1 to 10 minutes, using very low temperatures, for example less than −70° C. The combination of formulations can be frozen by any suitable method known in the art, but preferably it is cryogenically frozen.

The final step is lyophilisation of the frozen combination of formulations. Lyophilisation is also called freeze-drying. Preferably, the lyophilisation process is carried out in a freeze-drying chamber typically operating under vacuum of 0.1 to 1.0 mbar for a period of time from 180 to 500 minutes.

In a preferred embodiment, the single unit oral dose pharmaceutical composition has a weight ratio of calcitriol to naltrexone in the range of 1:1 to 10:1.

In a preferred embodiment, the single unit oral dose composition comprises a third active ingredient. Preferably, the third active ingredient is present in a third formulation. The third formulation can be made according to the methods described above for the first and second formulations.

In a preferred embodiment, the third active ingredient is a cannabinoid. Preferably, the cannabinoid is selected from the list consisting of: cannabidiol, cannabidiolic acid, cannabinol, tetrahydrocannabivarin, arachidonoylethanolamine, cannabidivarin, 2-arachidonoylglycerol, cannabigerol, cannabivarin, cannabichromene, 2-arachidonoyl glyceryl ether, N-arachidonoyl dopamine, virodhamine, dronabinol, nabilone, rimonabant or combinations thereof.

In a preferred embodiment, the third active is a flavonoid. Preferably, the flavonoid is a flavonoid found in Cannabis, such as cannaflavin-A, cannaflavin-B, cannaflavin-C, quercetin, isovitexin, apigenin, beta-sitosterol, luteolin, orientin, catechin, vitexin, silymarin, Kaempferol or combinations thereof.

In a preferred embodiment, the third active is a terpene. Preferably, the terpene is a terpene found in Cannabis, such as limonene, linalool, myrcene, pinene, phytol, terpinolene, trans-nerolidol, valencene, humulene, geraniol, eucalyptol, delta 3 carene, caryophyllene, camphene, borneol, bisabolol or combinations thereof.

In a preferred embodiment, the single unit oral dose pharmaceutical composition for use as a medicament for separate, sequential or simultaneous administration with an agonist of a 5-hydroxytryptamine (5-HT) receptor is for the treatment of an autoimmune disease, Alzheimer's disease, dementia, motor neuron disease (MND), depression, migraine, pain including chronic pain, psychosis, anxiety, irritability, obsessive compulsive disorder (OCD), fibromyalgia, post-traumatic stress disorder (PTSD), cluster headaches, paranoia, panic attacks, flashbacks, substance abuse, angina, obesity, parkinsonism, irritable bowel syndrome (IBS), malignant carcinoid syndrome; dyspepsia; non-cardiac chest pain; gastro-oesophageal reflux disease; diabetes or emesis such as cancer therapy-induced vomiting by cytotoxic drugs and radiation.

In a preferred embodiment the single unit oral dose pharmaceutical composition for use as a medicament for separate, sequential or simultaneous administration with an agonist of a 5-hydroxytryptamine (5-HT) receptor is for the treatment of a cognitive or neurological disorder.

In a preferred embodiment, the single unit oral dose pharmaceutical composition for use as a medicament is used for subjects having a reduced ability to metabolise vitamin D. This is because such subjects are unable to metabolise vitamin D in the normal way to form calcitriol. Certain cells have elevated CYP enzymes which means vitamin D metabolism is compromised—specifically, CCT cannot be converted from its precursor CCD as it is unable to be converted from vitamin D. Administration of calcitriol in a single unit oral dose according to the invention overcomes this problem.

In a preferred embodiment, the single unit dose pharmaceutical composition is to be administered to the subject in a first treatment phase, and wherein after the first treatment phase, the subject is administered the agonist of a 5-HT receptor in a subsequent second treatment phase.

As used herein, the term “subject” refers to any animal (for example, a mammal), including, but not limited to, humans, non-human primates, canines, felines, rodents, and the like, which is to be the recipient of a treatment in which a composition comprising an agonist of the 5-hydroxytryptamine (5-HT) receptor is to be used according to the present invention. Typically, the terms “subject” and “patient” are used interchangeably herein in reference to a human subject.

### EXAMPLES

### Example 1: Synthesis of a Single Unit Dose According to the Invention

A single unit dose pharmaceutical composition according to a preferred embodiment of the invention was made as follows:

A tablet containing naltrexone (LDN) was formulated with the following components:

| | Quantity | Quantity | | |
|---|---|---|---|---|
| | per low | per high | | |
| | strength | strength | | |
| | (1 mg) | (10 mg) | | Reference |
| Component | Tablet (mg) | Tablet (mg) | Function | to Standard |
| Naltrexone | 1.00 | 10.00 | Active | Ph |
| Hydrochloride¹ | | | | Eur, USP |
| Microcrystalline | 91.30 | 82.30 | Filler | Ph. Eur., JP, |
| Cellulose | | | | USP/NF |
| Pregelatinised | 5.00 | 5.00 | Filler | Ph. Eur., |
| Starch | | | | USP/NF |
| Crospovidone | 2.00 | 2.00 | Disintegrant | Ph. Eur., JP, |
| | | | | USP/NF |
| Silica, Colloidal | 0.20 | 0.20 | Glidant | Ph. Eur., |
| Anhydrous | | | | USP/NF |
| Magnesium | 0.50 | 0.50 | Lubricant | Ph. Eur., JP, |
| Stearate | | | | USP/NF |
| Total per tablet | 100.00 | 100.00 | | |

The formulation was initially a dry mix which was pressed into a 100 mg tablet. The tablet has a crystalline coating, onto which the CCT formulation is attached.

The CCT formulation was formulated from a liquid solution or suspension containing 140 ug CCT, gelatin and mannitol.

The liquid/suspension was added to pre-formed blisters and then the above described LDN tablet was attached to form the single unit dose. It was then passed through a cryogenic freezing process. The frozen units then undergo lyophilisation process in freeze dryers. The blisters containing the dried units are then sealed via a heat-seal process to protect the product from varying environmental conditions and ensure long-term stability.

### Example 2

CD3+ lymphocytes were isolated from the blood of haematologically normal volunteers. Cells placed into 25 cm³ flasks at a concentration of 1×10⁶/ml, and left to equilibrate in an incubator with a humidified atmosphere and 5% CO₂ in air at 37° C. Cells were then treated for 4 h with naltrexone (NTX) (10 uM) or low dose naltrexone (LDN) (10 nM). RNA extraction was performed using Trizol according to standard procedures, before being processed for microarray analysis according to the standard methodologies. Briefly, equal amounts of biotinylated cRNA was hybridised to the Illumina human HT12-v3 arrays for 18 h and subsequently processed according to manufacturer's instructions before scanning on an Illumina BeadArray Reader. The image data were processed using default values in GenomeStudio v2009.1 with imputation of missing data, before loading onto GeneSpring v9.0 for data normalisation and filtering. Data analyses were performed using Excel software, and the ratio of gene expression signals in treated vs. untreated calculated.

The results are shown below in Table 1.

TABLE 1

| Probe | | | | | | NTX/ | LDN/ |
|---|---|---|---|---|---|---|---|
| ID | Definition | Symbol | UN | NTX | LDN | UN | UN |
| ILMN_{—} | 5-HT receptor | HTR1E | 93.05 | 95.70 | 101.20 | 1.03 | 1.09 |
| 1794095 | 1E, mRNA. | | | | | | |
| ILMN_{—} | 5-HT receptor | HTR2A | 80.00 | 85.30 | 80.00 | 1.07 | 1.00 |
| 1715496 | 2A, mRNA. | | | | | | |
| ILMN_{—} | 5-HT receptor | HTR2B | 111.95 | 177.95 | 147.45 | 1.59 | 1.32 |
| 1735764 | 2B, mRNA. | | | | | | |
| ILMN_{—} | 5-HT receptor | HTR2C | 80.00 | 89.50 | 85.50 | 1.12 | 1.07 |
| 1801357 | 2C, mRNA. | | | | | | |
| ILMN_{—} | 5-HT receptor | HTR3A | 80.00 | 80.60 | 82.35 | 1.01 | 1.03 |
| 1662070 | 3A, transcript | | | | | | |
| | variant 2, | | | | | | |
| | mRNA. | | | | | | |
| ILMN_{—} | 5-HT receptor | HTR3A | 85.10 | 86.75 | 94.00 | 1.02 | 1.10 |
| 1681492 | 3A, transcript | | | | | | |
| | variant 1, | | | | | | |
| | mRNA. | | | | | | |
| ILMN_{—} | 5-HT receptor | HTR3A | 100.55 | 151.75 | 124.70 | 1.51 | 1.24 |
| 2371079 | 3A, transcript | | | | | | |
| | variant 2, | | | | | | |
| | mRNA. | | | | | | |
| ILMN_{—} | 5-HT receptor | HTR4 | 90.35 | 96.10 | 100.20 | 1.06 | 1.11 |
| 1676350 | 4, transcript | | | | | | |
| | variant a, | | | | | | |
| | mRNA. | | | | | | |
| ILMN_{—} | 5-HT receptor | HTR4 | 80.00 | 88.20 | 93.50 | 1.10 | 1.17 |
| 1808258 | 4, transcript | | | | | | |
| | variant hb, | | | | | | |
| | mRNA. | | | | | | |
| ILMN_{—} | 5-HT receptor 7 | HTR7 | 80.00 | 86.60 | 80.00 | 1.08 | 1.00 |
| 1744217 | (adenylate | | | | | | |
| | cyclase- | | | | | | |
| | coupled), | | | | | | |
| | transcript | | | | | | |
| | variant a, | | | | | | |
| | mRNA. | | | | | | |

As can be seen from the above data, both higher dose and low dose naltrexone increase the levels of mRNA for one or more of 5-HT receptor proteins.

1. A single unit oral dose pharmaceutical composition for use as a medicament for separate, sequential or simultaneous administration with an agonist of a 5-hydroxytryptamine (5-HT) receptor;
wherein the single unit oral dose pharmaceutical composition comprises a first active ingredient and a second active ingredient;
wherein the first active ingredient is for absorption in the gastrointestinal tract from the oesophagus onwards and the second active ingredient is for absorption in the oral cavity; and
wherein the first active ingredient is naltrexone in an amount of 0.01 to 10 mg and the second active ingredient is calcitriol in an amount of 80 to 200 ug.

2. The single unit oral dose pharmaceutical composition for use according to claim 1, wherein the 5-HT receptor is 5-HT receptor subtype 2A, 2B or 2C.

3. The single unit oral dose pharmaceutical composition for use according to claim 1, wherein the 5-HT receptor is 5-HT receptor subtype 3.

4. The single unit oral dose pharmaceutical composition for use according to claim 1, wherein the agonist of a 5-HT receptor is selected from the group consisting of psilocybin, psilocin, mescaline, lysergic acid diethylamide (LSD), ketamine, salvinorin A, ibotenic acid, muscimol, N,N-dimethyltryptamine (DMT), 3,4-methylenedioxymethamphetamine (MDMA), methyldiethanolamine, also known as N-methyl diethanolamine (MDEA), 3,4-methylenedioxy amphetamine (MDA), 4-Bromo-2,5-dimethoxyphenethylamine (2C-B); 1-(8-Bromo-2,3,6,7-tetrahydrobenzo-[1,2-b;4,5-b′]difuran-4-yl)2-amino-ethane (2C-B-fly); 4-Ethyl-2,5-dimeth-oxyphenethylamine (2C-E); 4-Ethyl-thio-2,5-dimethoxyphenethylamine (2C-T-2); 4-Ethylthio-2,5-dimethoxy-amphetamine (ALEPH-2); 4-Ethylthio-2,5-dimethoxyphenylbutylamine (4C-T-2); 2,5-Dimethoxy-4-ethoxyamphetamine (MEM); 2,4,5-Trimethox-amphetamine (TMA-2); 3,4,5-Trimethoxamphetamine (TMA); 2,5-Dimethoxy-4-bromo-amphetamine (DOB); 2,5-Dimethoxy-4-iodo-amphetamine (DOI); 2,5-Dimethoxy-4-methylamphetamine (DOM); 2,5-Dimethoxy-4-ethylamphet-amine (DOET); 5-Methoxy-N,N-dimethyl-tryptamine (5-MeO-DMT); N,N-Dipropyltryptamine (DPT); 5-Methoxy-N-methyl-N-isopropyltryptamine (5-MeO-MIPT); N,N-Diisopropyltryptamine (DIPT); 5-Methoxy-N,N-diiso-propyltryptamine (5-MeO-DIPT); 6-Fluoro-N,N-dimethyltryptamine (6-fluoro-DMT); lisuride; ibogaine; cis-2a; RR-2b; SS-2c; 2-Ethyl-5-methoxy-N,N-dimethyltryptamine (EMDT), serotonin hydrochloride, or metabolites thereof and combinations thereof.

5. The single unit oral dose pharmaceutical composition for use according to claim 1, wherein the agonist of a 5-HT receptor is selected from the group consisting of m-chlorophenylbiguanide hydrochloride, N-methylquipazine dimaleate, PSEM 895, quipazine dimaleate, RS56812 hydrochloride, serotonin hydrochloride, SR7227 hydrochloride, 1-phenylbiguanide hydrochloride, cereulide, 2-methyl-5-HT, alpha-methyltryptamine, bufotenin, chlorophenylbiguanide, ibogaine, varenicline, YM-31636, and metabolites thereof.

6. The single unit oral dose pharmaceutical composition for use according to claim 1, comprising 1 mg to 6 mg of naltrexone.

7. The single unit oral dose pharmaceutical composition for use according to claim 1, comprising the naltrexone in a first formulation and the calcitriol in a second formulation.

8. The single unit oral dose pharmaceutical composition for use according to claim 7, wherein the first formulation comprising naltrexone is formulated as a tablet, a capsule, powder, a disc, a caplet, granules or pellets.

9. The single unit oral dose pharmaceutical composition for use according to claim 7, wherein the second formulation comprising calcitriol is formulated for sublingual absorption.

10. The single unit oral dose pharmaceutical composition for use according to claim 9, wherein the second formulation comprising calcitriol is formulated as a liquid solution, suspension or emulsion which has been frozen and lyophilised.

11. The single unit oral dose pharmaceutical composition for use according to claim 7, wherein the first formulation comprising naltrexone is in the form of a tablet and wherein the second formulation comprising calcitriol is a lyophilised and frozen liquid solution, suspension or emulsion which is attached to the tablet.

12. The single unit oral dose pharmaceutical composition for use according to claim 1, wherein the composition, is for combined therapy with the 5-HT agonist of a cognitive or neurological disorder.

13. The single unit oral dose pharmaceutical composition according to claim 1 for use in the treatment of an autoimmune disease, Alzheimer's disease, dementia, motor neuron disease (MND), depression, migraine, pain including chronic pain, psychosis, anxiety, irritability, obsessive compulsive disorder (OCD), fibromyalgia, post-traumatic stress disorder (PTSD), cluster headaches, paranoia, panic attacks, flashbacks, substance abuse, angina, obesity, parkinsonism, irritable bowel syndrome (IBS), malignant carcinoid syndrome; dyspepsia; non-cardiac chest pain; gastro-oesophageal reflux disease; diabetes or emesis such as cancer therapy-induced vomiting by cytotoxic drugs and radiation.

14. The single unit oral dose pharmaceutical composition for use according to claim 1, wherein the single unit dose pharmaceutical composition is to be administered to the subject in a first treatment phase, and wherein after the first treatment phase, the subject is administered the agonist of a 5-HT receptor in a subsequent second treatment phase.

15. The single unit dose pharmaceutical composition for use according to claim 1, wherein the weight ratio of calcitriol to naltrexone is in the range of 1:1 to 10:1.

16. The single unit dose pharmaceutical composition for use according to claim 1, further comprising a cannabinoid, flavonoid or terpene.

17. The single unit oral dose pharmaceutical composition for use according to claim 1, comprising 3 to 4.5 mg of naltrexone.

18. The single unit oral dose pharmaceutical composition for use according to claim 8, wherein the second formulation comprising calcitriol is formulated for sublingual absorption.

19. The single unit dose pharmaceutical composition for use according to claim 16, wherein the cannabinoid, flavonoid or terpene is selected from the list consisting of: cannabidiol, cannabidiolic acid, cannabinol, tetrahydrocannabivarin, arachidonoylethanolamine, cannabidivarin, 2-arachidonoylglycerol, cannabigerol, cannabivarin, cannabichromene, 2-arachidonoyl glyceryl ether, N-arachidonoyl dopamine, virodhamine, dronabinol, nabilone, rimonabant, cannaflavin-A, cannaflavin-B, cannaflavin-C, quercetin, isovitexin, apigenin, beta-sitosterol, luteolin, orientin, catechin, vitexin, silymarin, Kaempferol, limonene, linalool, myrcene, pinene, phytol, terpinolene, trans-nerolidol, valencene, humulene, geraniol, eucalyptol, delta 3 carene, caryophyllene, camphene, borneol, bisabolol or combinations thereof.
